(19) United States
(12) Patent Application Publication
(10) Pub. No.: US 2017/0224827 A1
(43) Pub. Date: Aug. 10, 2017

(54) STABLE PRESERVATIVE FREE OPHTHALMIC FORMULATIONS OF OPIOID ANTAGONISTS

(71) Applicant: IMUNEKS FARMA ILAC SANAYI VE TICARET A.S., Gayrettepe/Istanbul (TR)

(72) Inventors: Mehmet Nevzat Pisak, Gayrettepe/Istanbul (TR); Cengiz Celayir, Beykoz/Istanbul (TR)

(73) Assignee: IMUNEKS FARMA ILAC SANAYI VE TICARET ANONIM SIRKET, Istanbul (TR)

(21) Appl. No.: 15/328,781

(22) PCT Filed: Jul. 16, 2015
(86) PCT No.: PCT/IB2015/055384

§ 371 Date: Jan. 24, 2017

(30) Foreign Application Priority Data
Jul. 25, 2014 (TR) PCT/TR2014/000303

(51) Int. Cl. A61K 47/38; A61K 47/02; A61K 47/22; A61K 9/00; A61K 31/485

(52) U.S. Cl. CPC A61K 47/38; A61K 9/0048; A61K 31/485; A61K 47/22; A61K 47/02

(57) ABSTRACT

The present application is directed to stable preservative free ophthalmic formulations of opioid antagonists and use thereof in the treatment of retinal degenerative diseases.

### FIELD OF THE INVENTION

The present application is directed to stable preservative free ophthalmic formulations of opioid antagonists and use thereof in the treatment of retinal degenerative diseases.

### BACKGROUND OF THE INVENTION

Current treatment efforts for macular degeneration and retinitis pigmentosa have been very poor at best; there are daily supplements and intraocular injections with serious side effects available for the patients but these treatment methods have not been very successful as a treatment and they are merely used to stop the progression of the disease rather than reversing it's devastating effect: complete vision loss.

The use of opioid antagonists such as naltrexone, naloxone or nalmefene as active substances, as a stable preservative free solution is aimed for the treatment of retinal degenerative diseases, mainly for the treatment of macular degeneration and retinitis pigmentosa. The stable preservative free solution preferably comprises naltrexone as active substance in low doses. Opioid antagonists may be in the form of any pharmaceutically acceptable salt thereof.

The treatment within the context of this invention also refers to prophylactic and/or preventative application of the stable preservative free opioid antagonist composition. The desired result is to completely treat, and prevent or slow down the progression of the retinal degenerative diseases or conditions.

Low doses of naltrexone exerts an opiate blocking action substantially exclusively for Mu opiate receptors over Delta opiate receptors. That is the prescribed low dose regimen such that naltrexone does exert a substantial blocking action for Mu opiate receptors but does not exert such an action against other opiate receptors.

The brief blockade of opioid receptors sites by using low dose naltrexone (LDN) once daily is believed to produce a prolonged up-regulation of the body's immune system by inducing an increase in endogeneous opioid peptides production. Normal volunteers who have followed LDN regimen have been found to have higher levels of beta-endorphins in their blood in the following days.

Low dose naltrexone methodology is targeting modulatory effects of endogeneous opioid peptides on the body's immune system. It seems that the therapy primarily opposes the complement cascade elimination process by MAC activation. Such an immune system attack causes the accumulation of extracellular debris in the macula consequently leading to drusen formation. However, other immune system related biochemical mechanisms may as well come to play to explain the observed therapeutic action.

Treatment of macular degeneration and retinitis pigmentosa with the eye drops of naltrexone is a promising treatment. However, naltrexone has stability problems due to photo-induced oxidation and hydrolysis, which cause the degradation of naltrexone in the eye drops.

Ophthalmic preparations comprising any opioid antagonist for use in the treatment of macular degeneration and retinitis pigmentosa; and any technical solution for the stability problem of the ophthalmic preparations comprising naltrexone have not been disclosed in the prior art.

U.S. Pat. No. 6,110,926A relates to the aqueous eye drop composition consisting essentially of water with naloxone hydrochloride and, as stabilizing agent, a polyhydric alcohol dissolved therein. The invention embraces the use of such solutions in opioid addiction testing.

US20120101033A1 relates to the treatment method of retinitis pigmentosa with the use of ophthalmic drops or preparations comprising insulin, IGF-1 and chlorin e6. Said method comprises the use of compatible preservatives such as e.g., benzalkonium chloride.

EP2441453A1 relates to the pharmaceutical composition for treatment or prophylaxis of eye pathologies comprising mitochondria-addressed antioxidant, pH buffer, concentration stabilizer of mitochondrial antioxidant, prolongator (thickener), isotonic component, preservative and additional active ingredient. A study of stability disclosed in the patent shows that addition of 0.01% benzalkonium chloride prevents a decrease in the concentration of active ingredient during storage for 24 hours at room temperature.

US20130281529A1 relates to the eye drop preparation comprising an ophthalmic solution composition comprising latanoprost; a nonionic surfactant; organic amine and/or a sugar or a derivative thereof; and boric acid and/or phosphoric acid in certain amounts for use in the treatment of glaucoma. According to the invention, it is believed that only the combination of these components in given amounts results in sufficient stability.

WO2008074885A2 relates to the processes for manufacturing pre-concentrates of ophthalmic oil-in-water emulsions by diluting the pre-concentrate. The diluting aqueous solution of the invention comprises benzalkonium chloride, the most commonly used preservative in ophthalmology.

CA2625568C relates to the cationic ophthalmic oil-in-water type emulsion, which comprises colloid particles having an oily core surrounded by an interfacial film, said emulsion comprising at least one cationic agent and at least one non ionic surfactant, said oily core comprising a drug, particularly latanoprost, and said emulsion being free of the water-soluble polymers selected from the group consisting of a polyvinyl compound, a water-soluble cellulose compound and a polysaccharide. It is believed that since the prostaglandin is solubilized in the oily core of the emulsion, it is less available to contact with agents enhancing its degradation.

WO2004062660A1 relates to the ophthalmic composition which is comfortable to the human eye comprising hyaluronic acid; stabilized oxy-chloro complex; and boric acid/borate buffer in certain amounts; wherein the composition comprises no more than about 0.0075% hydrogen peroxide. Stabilized oxy-chloro complex is used for its preservative efficacy.

WO1993023010A1 relates to the ophthalmic composition comprising a cyclosporin and a surfactant selected from polyoxyethylene fatty acid esters, polyoxyethylene alkylphenyl ethers and polyoxyethylene, alkyl ethers, or mixtures thereof. It is believed that this combination provides higher stability.

EP0025202A2 relates to the ophthalmic solution for the adjustment of intraocular pressure comprising an ophthalmologically acceptable, water-soluble salt of 2-acetyl-7-(2-hydroxy-3-isopropylaminopropoxy)benzofuran; benzalkonium chloride or benzethonium chloride as preservative; and at least one viscosity inducing agent, the solution being adjusted to pH 5.0 to 8.0 with a buffer agent.

Consequently, ophthalmic preparations comprising any opioid antagonist, particularly naltrexone for use in the treatment of macular degeneration and retinitis pigmentosa; and any technical solution for the stability problem of the ophthalmic preparations comprising naltrexone have not been disclosed in the prior art. Moreover, ophthalmic compositions in the prior art suggest the use of preservatives including surfactants, stabilizing agents, and the like.

However, eye drops containing preservatives have been known to cause tear film instability, loss of goblet cells, conjunctival squamous metaplasia and apoptosis, disruption of the corneal epithelium barrier, and damage to deeper ocular tissues, which may lead to foreign body sensation, dryness in the eyes, burning and irritation in the eyes, ocular surface breakdown, and dryness.

Eye drops free of preservatives create much fewer incidences of irritation in the eyes mainly; pain or discomfort, foreign body sensation, stinging or burning, and dry eye sensation.

The main preservative that is used in the eye drop solutions is benzalkonium chloride, side effects occur in patients that have sensitivity reactions to benzalkonium chloride. So an important portion of the patient population suffering from retinal degenerative diseases will not be able to use a solution comprising naltrexone as eye drops if it contains a preservative. Another drawback of preservatives is that: “Preservative-induced adverse effects are far from being restricted to only allergic reactions, and side effects are often very difficult to identify because they mostly occur in a delayed or poorly specific manner” [Baudouin C, Labbé A, Liang H, Pauly A, Brignole-Baudouin F. “Preservatives in eyedrops: the good, the bad and the ugly.” Prog Retin Eye Res. 2010 July; 29(4): 312-34.].

In order to minimize the incidence of any possible side effects resulting from the long term treatment of macular degeneration, a preservative free naltrexone solution in the form of eye-drops is needed. This will be a revolutionary treatment with minor or probably no side effects, taking into consideration the good safety profile of naltrexone for more than 30 years in systemic use. Moreover, the stability of the preservative free ophthalmic compositions of naltrexone is also desired. Both stable and preservative free ophthalmic compositions of naltrexone have never been disclosed in the prior art. Common approaches for preservative free ophthalmic compositions are directed to develop a packaging system that maintains the sterility of the preservative free products throughout its shelf-life and during use. These alternatives usually lead to high cost of manufacture, are not feasible and are not applicable in terms of preservative free ophthalmic naltrexone compositions.

Therefore, there is still a need to formulate a stable preservative free ophthalmic composition of opioid antogonists.

### SUMMARY OF THE INVENTION

The present invention relates to an ophthalmic composition comprising an opioid antagonist, at least one polysaccharide and/or at least one buffer, said composition being preservative free.

### DETAILED DESCRIPTION OF THE INVENTION

The present invention is directed to stable preservative free aqueous ophthalmic formulations of opioid antagonists, preferably of naltrexone as the active ingredient. The formulation of an eye drop solution comprising an opioid antagonist is preferable for patients in several aspects: higher safety, less side effects, and most importantly because the patients will not have the associated side effects; increased patient compliance and a successful treatment.

Preservative free solution of an opioid antagonist for ophthalmic use, is a sterile, isotonic solution. The active ingredient of the drug formulation is preferably naltrexone or a pharmaceutically acceptable salt thereof, more preferably naltrexone HCl.

Surprisingly, it has been found that preservative free ophthalmic compositions of the invention comprising naltrexone, at least one polysaccharide and/or at least one buffer present the highest stability, absorption and safety. Ophthalmic compositions of the invention being preservative free and comprising at least one polysaccharide preferably in an amount of 0.05-10% w/v and/or at least one buffer preferably in an amount of 0.002-1% w/v have lead to ophthalmic compositions having increased stability, absorption and safety.

At least one polysaccharide used in the invention may be, but is not limited to, cellulose derivatives (methyl cellulose, hydroxyethyl cellulose, hydroxypropylmethyl cellulose, etc.), mannitol, chitosan, xyloglucan, arabinogalactan, hyaluronic acid, alginic acid, gellan gum, and the like. The polysaccharide used in the invention is preferably a cellulose derivative, more preferably hydroxyethyl cellulose, and it has been demonstrated that the use of a cellulose derivative preferably in an amount of 0.05-10% w/v, more preferably in an amount of 0.2-0.5% w/v increases the stability and absorption. The use of hydroxyethyl cellulose is more preferable. Mannitol in an amount of 0.05-10% w/v is also preferable according to the invention.

Hydroxypropylmethyl cellulose, a cellulose derivative commonly used in formulations for ophthalmic use, has been known to create a film layer providing sustained release of the drug from the formulation (Bhowmik, M. et. al. “Methyl Cellulose Based Sustained Release Thermosensitive in situ Fast Gelling Ocular Delivery of Ketorolac Tromethamine” Asian Journal of Chemistry Vol. 22, No. 3 (2010), 2147-2154). However, although hydroxyethyl cellulose also creates a film layer that increases the stability and absorption of naltrexone, it does not create a sustained release formulation that might affect the mechanism of action of low dose naltrexone which is based on the temporary inhibition of the opioid receptors.

At least one buffer used in the invention may be, but is not limited to, citric acid (sodium citrate heptahydrate, sodium citrate monobasic, or combination thereof), phosphoric acid (sodium phosphate dibasic heptahydrate, sodium phosphate monobasic, or combination thereof), boric acid, and the like. Citrate buffers are preferred in the invention and it has been demonstrated that the use of citrate buffers preferably in an amount of 0.002-1% w/v, more preferably in an amount of 0.01-0.5% w/v increases the stability and provides the desired pH value of the solution and diminishes the risk for calcification of the cornea.

Tonicity adjustment agents are also included. An agent for tonicity used in the invention may be, but is not limited to, sodium chloride, propylene glycol, potassium chloride, calcium chloride, mannitol and the like.

Sterile water (water for injection) is used to create an ideal medium for the water soluble active ingredient. Sterile water used in the invention also eliminates the risk of infections.

In order not to cause any irritation in the eyes, the pH value of the solution would be between 6 to 8, preferably 6.8 and the osmolality would be preferably between 270-350 mOsmol/kg.

The naltrexone composition of the invention may be in the form of a solution, emulsion, dispersion, suspension, ointment, reverse emulsion and microemulsion. The naltrexone composition of the invention is preferably in the solution form. Said solution according to the invention may be contained in a unit dose form. Said solution according to the invention may be contained in a multi dose form.

The naltrexone composition of the invention is useful for treating macular degeneration and retinitis pigmentosa; and is applied once or twice a day to each eye. The naltrexone composition of the invention has greater bioavailability of naltrexone in the eye of the patient with fewer side effects than the naltrexone compositions preserved with benzalkonium chloride or any other preservatives.

The following examples provide a detailed illustration of compositions of the present invention. However, they are illustrative only and not intented to limit the scope of the invention.

### EXAMPLE 1

### A Preservative Free Ophthalmic Naltrexone Solution Composition

| Ingredient | Function | Amount (% w/v) |
|---|---|---|
| Naltrexone HCl | Active substance | 0.001-50 |
| Sodium citrate heptahydrate | Buffer | 0.2-0.4 |
| Sodium citrate monobasic | Buffer | 0.01-0.05 |
| Hydroxyethyl cellulose | Absorption enhancer | 0.05-1 |
| Sodium chloride | Tonicity agent | 0.3-0.9 |
| Sterile water (water for | Carrier | Quantity sufficient |
| injection) | | |

Said preservative free naltrexone composition has a pH of about 6 to 8 and osmolality preferably between 270-350 mOsmol/kg.

### EXAMPLE 2

### A Preservative Free Ophthalmic Naltrexone Solution Composition

| Ingredient | Function | Amount (% w/v) |
|---|---|---|
| Naltrexone HCl | Active substance | 1.8 |
| Sodium citrate heptahydrate | Buffer | 0.3 |
| Sodium citrate monobasic | Buffer | 0.04 |
| Hydroxyethyl cellulose | Absorption enhancer | 0.32 |
| Sodium chloride | Tonicity agent | 0.78 |
| Sterile water (water for | Carrier | Quantity sufficient |
| injection) | | |

Said preservative free naltrexone composition has a pH of about 6 to 8 and osmolality preferably between 270-350 mOsmol/kg.

### EXAMPLE 3

### A Preservative Free Ophthalmic Naltrexone Solution Composition without Buffer

| | | | |
|---|---|---|---|
| Naltrexone HCl | 100 | mg | Active substance |
| Tocophersolan | 0.5 | g | Antioxidant |
| Hydroxyethyl cellulose | 0.32 | g | Viscosity enhancing agent |
| Sodium chloride | 0.92 | g | Tonicity agent |
| Sterile water (water for | q.s. 100 | mL | Carrier |
| injection) | | | |

### EXAMPLE 4

### A Preservative Free Ophthalmic Naltrexone Solution Composition without Buffer

| | | | |
|---|---|---|---|
| Naltrexone HCl | 100 | mg | Active substance |
| Tocophersolan | 0.5 | g | Antioxidant |
| Mannitol | 5 | g | Tonicity agent |
| Sterile water (water for | q.s. 100 | mL | Carrier |
| injection) | | | |

The present invention entails preservative free ophthalmic naltrexone solution formulations in unit-dose or multi-dose form having increased stability, absorption and safety. The results achieved with a preservative free opioid antagonist formulation will be less side effects such as foreign body sensation in the eyes, dryness in the eyes, burning and irritation in the eyes, and ocular surface breakdown, consequently increased patient compliance and the success rate of the treatment. Said preservative free formulations of the invention comprising naltrexone have higher stability, absorption and bioavailability. All of the above mentioned details of the invention create a superior product with higher stability, greater efficacy, less side effects and higher rates of patient compliance hence most importantly a preferable treatment.

1. Ophthalmic composition comprising an opioid antagonist, at least one polysaccharide, and/or at least one buffer, said composition being preservative free.

2. Ophthalmic composition according to claim 1, characterized in that said composition comprises an opioid antagonist, at least one polysaccharide in an amount of 0.05-10% w/v and/or at least one buffer in an amount of 0.002-1% w/v, said composition being preservative free.

3. Ophthalmic composition according to claim 1, characterized in that said composition comprises naltrexone or a pharmaceutically acceptable salt thereof as the opioid antagonist.

4. Ophthalmic composition according to claim 1, characterized in that said composition comprises a cellulose derivative as the polysaccharide.

5. Ophthalmic composition according to claim 4, characterized in that said composition comprises hydroxyethyl cellulose as the cellulose derivative.

6. Ophthalmic composition according to claim 1, characterized in that said composition comprises mannitol as the polysaccharide.

7. Ophthalmic composition according to claim 1, characterized in that said composition comprises sodium citrate heptahydrate and sodium citrate monobasic as the buffer.

8. Ophthalmic composition according to claim 1, characterized in that said composition further comprises a tonicity agent.

9. Ophthalmic composition according to claim 8, characterized in that said composition further comprises sodium chloride as the tonicity agent.

10. Ophthalmic composition according to claim 8, characterized in that said composition comprises mannitol as the tonicity agent.

11. Ophthalmic composition according to claim 1, characterized in that said composition further comprises a carrier.

12. Ophthalmic composition according to claim 11, characterized in that said composition further comprises sterile water as the carrier.

13. Ophthalmic composition according to claim 1, characterized in that said composition comprises naltrexone HCl in an amount of 0.001-50% w/v, sodium citrate heptahydrate in an amount of 0.2-0.4% w/v, sodium citrate monobasic in an amount of 0.01-0.05% w/v, hydroxyethyl cellulose in an amount of 0.05-1% w/v, sodium chloride in an amount of 0.3-0.9% w/v and sterile water.

14. Ophthalmic composition according to claim 1, characterized in that said composition comprises naltrexone HCl in an amount of 100 mg, tocophersolan in an amount of 0.5 g, hydroxyethyl cellulose in an amount of 0.32 g, sodium chloride in an amount of 0.92 g and sterile water in an amount of 100 ml.

15. Ophthalmic composition according to claim 1, characterized in that said composition comprises naltrexone HCl in an amount of 100 mg, tocophersolan in an amount of 0.5 g, mannitol in an amount of 5 g and sterile water in an amount of 100 ml.

16. Ophthalmic composition according to any of the claims 1 to 15, characterized in that said composition is in the form of a sterile solution.

17. Ophthalmic composition according to any of the claims 1 to 16, characterized in that said composition is contained in a unit dose form.

18. Ophthalmic composition according to any of the claims 1 to 17, characterized in that said composition is contained in a multi dose form.

19. Ophthalmic composition according to any of the claims 1 to 18 for use in the treatment of retinal degenerative diseases, preferably in the treatment of macular degeneration and retinitis pigmentosa.

20. Ophthalmic composition according to claim 19 for use in the treatment of retinal degenerative diseases, preferably in the treatment of macular degeneration and retinitis pigmentosa once or twice a day.

21. Ophthalmic composition according to claim 16, characterized in that the pH value of the solution is between 6 to 8 and the osmolality is between 270-350 mOsmol/kg.

22. Ophthalmic composition according to claim 1, characterized in that the composition is in the form of a solution, emulsion, dispersion, suspension, ointment, reverse emulsion and microemulsion.
